(19) United States
(12) Patent Application Publication
(10) Pub. No.: US 2006/0228333 A1
(43) Pub. Date: Oct. 12, 2006

(54) Methods for control of tumors and chronic infections by modulating immunologically informed carriers homing to tolerogenic organs or tissues

(76) Inventor: Kye-Hyung Paik, Rancho Santa Fe, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/254,063

(22) Filed: Oct. 18, 2005

Related U.S. Application Data
(60) Provisional application No. 60/620,711, filed on Oct. 20, 2004.

(51) Int. Cl. A61K 39/395; A61K 38/21; A61K 31/557

(52) U.S. Cl. 424/85.7; 424/144.1; 514/573

(57) ABSTRACT

Methods for treating a patient after transplantation of a liver are presented which facilitate acceptance of the donor liver and treat the underlying disease or disorder that led to the transplant. A patient with a chronic viral infection is treated by inducing tolerance to the donor liver and suppressing tolerance of the body to the virus. Similarly, a patient with cancer is treated by inducing immunotolerance of a donor liver and suppressing immunotolerance of the cancer.

### FIELD OF THE INVENTION

This application claims the benefit of priority of U.S. Provisional application Ser. No. 60/620,711, filed Oct. 20, 2004, the entire contents of which is incorporated herein by reference.

The present invention relates generally to the area of organ transplantation, and more specifically to methods for increasing the acceptance of donor organs and reducing immunotolerance to underlying disease states, since immunotolerance has a significant involvement in the pathogenesis of certain diseases, such as chronic viral infections or tumors.

### BACKGROUND OF THE INVENTION

Two kinds of effector mechanisms mediate immune responses. Some immune responses are mediated by specific molecules, called antibodies, that are carried in the blood and lymph. The synthesis of antibodies occurs in a subset of lymphocytes called B lymphocytes or B cells. Antibody-mediated immunity is called humoral immunity. Other immune responses are mediated by cells. All of the leukocytes (white cells) of the blood participate in cell-mediated immunity (“CMI”). However, the specificity of the response depends upon a subset of lymphocytes called T lymphocytes or T cells. Most immune responses involve the activity and interplay of both the humoral and the cell-mediated branches of the immune system. See U.S. Pat. No. 5,910,309.

Immunotolerance is the mechanism by which a mammal does not produce an immune response to a particular antigen. In the best known example, a mammal does not produce an immune response to its own “self” antigens but recognizes and produces an appropriate immune response to foreign “non-self” antigens. See vonBoehmer, H., Annu. Rev. Immunol. 8:531 (1990). However, immunotolerance can develop for foreign antigens as well, such as in the case of chronic viral infection.

Immunotolerance is mediated primarily by T cells (e.g., CD4⁺ and CD8⁺ T cells) which become tolerized by exposure to self-antigen. Inside the thymus, one of the tolerogenic organs, T cells that are reactive to self-antigens are physically deleted from the cell population or made immunologically nonresponsive (e.g., anergy or indifference). Self-antigens expressed outside of the thymus usually induce tolerance by anergy or downregulation of cell surface molecules such as T-cell receptors. See Pircher, H. et al., Nature 351:482-485 (1991); Oehen, S. U. et al., Cell. Immunol. 158:342-352 (1994). B cells also can be tolerized by exposure to antigen, leading to elimination or functional nonresponsiveness of the B cells.

Transgenic animals have been used to investigate the mechanisms of immunotolerance because the foreign transgenes are expressed and recognized as self-antigens in the transgenic animal. Mice transgenic for various viral proteins have been used as immunotolerance models for investigating its relationship to human pathological conditions. The viral proteins are effectively self-antigens that can be readily identified and manipulated in vivo and in vitro. See Zinkernagel, R. M. et al., Immunol. Rev. 122:133-171 (1991).

Immunotolerance to a foreign antigen can be enhanced or induced in a mammal, for example by the use of immunosuppressive agents and radiation.

Immunosuppressive agents are used in solid organ transplantation as a means of protecting the newly implanted organ from destruction by the host's immune system. Optimally, the immunosuppressive agent is able to facilitate survival of the new organ while preserving the immunocompetence of the recipient against other foreign antigens. Typically, nonspecific immunosuppressive agents such as cyclosporine A, methotrexate, X-irradiation, steroids, FK506, cytotoxic drugs, and corticosteroids are used to prevent host rejection responses. Nonspecific immunosuppressive agents function by suppressing all aspects of the immune response, thereby greatly increasing a recipient's susceptibility to infections and diseases. The only known clinical condition in which complete systemic donor-specific transplantation tolerance occurs is when chimerism is created through bone marrow transplantation. See Qin et al., J. Exp. Med. 169:779 (1989); Sykes et al., Immunol. Today 9:23 (1988); Sharabi et al., J. Exp. Med. 169:493 (1989). This has been achieved in neonatal and adult animal models as well as in humans by total lymphoid irradiation of a recipient followed by bone marrow transplantation with donor cells.

Ultraviolet radiation is immunosuppressive. See Kripke, et al., J. Natl. Cancer Inst. 53:1333-1336 (1974). The immunosuppression induced by UV radiation is unique. Despite the limited ability of UV to penetrate tissue, the suppression seen following exposure to UV radiation is systemic. See Evertt et al., Photochem Photobiol 5:533-542 (1966). For example, after a single exposure to UV radiation, mice are unable to generate a delayed-type hypersensitivity reaction to antigens injected subcutaneously at a distant non-irradiated site. See Ullrich et al., Photochem Photobiol 43:633-638 (1986); Ullrich, et al., Transplantation 42:287-291 (1986); Molendijk et al., Immunology 62:299-305 (1987). The suppression is specific for the injected antigen, and associated with the appearance of splenic antigen-specific suppressor T lymphocytes. See Ullrich et al., Transplantation 46:115-119 (1988). Although it is not entirely clear how UV-irradiation of the skin can result in the induction of systemic immunosuppression, most of the evidence to date supports the concept that UV-induced soluble suppressive factors are involved.

A wide variety of soluble factors have been implicated in the induction of systemic suppression following UV exposure, including cis-urocanic acid, contra-IL-1, IL-1, prostaglandins, serum factors, and factors isolated from UV-irradiated keratinocytes. See De Fabo et al., J. Exp. Med 158:84-98 (1983); Schwarz et al., J. Immunol 138:1457-1463 (1987); Robertson et al., J. Invest Dermatol 88:380-387 (1987); Chung, H. T. et al., J. Immunol 137:2478-2484 (1986); Swartz, R. P, J. Invest. Dermatol 83:305-307 (1984); Harriott-Smith, T. G. et al., Clin Exp. Immunol 71:144-148 (1988); Schwarz, T. A. et al., J. Invest Dermatol 87:289-291 (1986); Kim, T. Y. et al., J Invest Dermatol 94:26-32 (1990); and Ullrich, S. E. et al., J Immunol 145:489-498 (1990).

Thymic injection presents another tool available to those of skill in the art for the purpose of inducing immunotolerance in a nonnatural host. Injection of class I MHC allopeptide-pulsed self dendritic cells can induce acquired thymic tolerance via the indirect allorecognition of foreign antigen in the thymus. This finding indicates that indirect antigen presentation by thymic antigen presenting cells is a critical component of antigen-specific immune responses. See Garrovillo, M., et al., Dept. of Surgery, Columbia University College of Physicians and Surgeons, 630 W. 168^{th} St., New York, N.Y. 10032. The anatomical distribution of various nonlymphoid cell types in the embryonic mouse thymus in vivo and in vitro, as well as in the thymic rudiment of the nude mouse embryo, has been studied. For this purpose a panel of monoclonal antibodies, directed to various types of stromal cells of the mouse thymus is used in combination with immunoperoxidase labeling on frozen sections. See Van Vliet E., et al. Eur J Immunol July; 15(7):675-81 (1985).

### SUMMARY OF THE INVENTION

According to one aspect of the invention, a method is provided for enhancing acceptance of a donor organ in a transplant patient with a chronic viral infection, such as chronic hepatitis A, B, C or D infection. The method comprises two steps: 1. administering an effective amount of an antiviral agent to the patient to break the immune tolerance to the virus which is a major part of the pathogenesis of the disease, and 2. administering an effective amount of an immunosuppressive agent to increase immune tolerance to the donor organ. The antiviral agent may be administered simultaneously with the immunosuppressive agent or they may be administered successively. In addition, treatment with the antiviral and/or immunosuppressive agents may begin before or after transplantation.

In one embodiment the antiviral agent is an interferon, preferably interferon alpha. In other embodiments the antiviral agent is an antibody specific for the virus. The use of antibodies may also prevent immunologically informed carriers from homing into tolerogenic organs/tissues.

In another aspect of the invention, a method is provided for enhancing acceptance of a donor organ transplant in a patient with cancer. The method preferably comprises administering an effective amount of an immunosuppressive agent to the patient and administering an anti-cancer agent to the patient. The anticancer agent may be administered simultaneously with the immunosuppressive agent or they may be administered successively. In addition, treatment with the anticancer and/or immunosuppressive agents may begin before or after transplantation.

In one embodiment the anticancer agent is an interferon, preferably alpha interferon. In other embodiments the anticancer agent is an antibody that recognizes the tumor.

In one embodiment the immunosuppressive agent is a T cell specific immunosuppressive agent, preferably rapamycin (RAPA).

The T cell specific agent may also be an antibody, such as an anti-CD4 or anti-CD8 antibody. In a particular embodiment, from about 6 months to about 10 years of treatment with the T cell specific agent is followed by treatment with a different immunosuppressive agent.

In other embodiments, one or more T cell specific immunosuppressive drugs with epithelial reaction are used, after controlling the immunotolerance of the disease.

In another embodiment the immunosuppressive agent is selected from the group consisting of Tacrolimus (FK 506), Cyclosporine A and Imuran. For example, in one embodiment a patient is treated with rapamycin for five years following transplantation, followed by treatment with tacrolimus for an additional five years.

In a further embodiment, donor cells are administered to the patient close to the time of the transplant. Donor cells are preferably peripheral blood mononuclear cells. From about 10⁸ to about 10⁹ donor cells per kg of body weight are preferably administered to the patient.

In another embodiment a prostaglandin is also administered to the patient following transplantation. Preferably the prostaglandin is administered for a period of at least about three weeks following transplantation. The prostaglandin is preferably selected from the group consisting of PGE1, PGE2, PGI1 and PGI2.

According to another embodiment, a nonsteroidal anti-inflammatory drug is administered to the patient following transplantation.

According to a further aspect, the invention provides a method of enhancing acceptance of a donor organ transplant in a patient comprising administering an immunosuppressive agent and interferon alpha to the patient. A prostaglandin and/or a non-steroidal anti-inflammatory drug may also be administered.

In a particular embodiment, a method of treating a patient suffering from HBV following a liver transplant is provided. Rapamycin or another immunosuppressive agent is administered following transplantation, preferably for a period of about five years. This may be followed by treatment with tacrolimus for an additional five years. Donor lymphocytes, preferably about 3×10⁸/kg, are injected into the patient and hepatitis B immune globulin is preferably administered. Rejection may be controlled with OKT3. After about 5 years, the patient is preferably treated with a hepatitis B vaccine.

In another embodiment, a patient suffering from a disease or disorder such as hepatitis C or a hepatoma is treated following a liver transplant. Rapamycin is preferably administered for a period of about 5 years. This may be followed by tacrolimus treatment for an additional five years. Donor lymphocytes, preferably about 3×10⁸/kg, are injected into the patient. PGE1, preferably about 32 ug/kg/day, is administered to the patient for about 3 weeks. Preferably, this treatment is followed by oral administration of 200 mg PGE 1 orally about three times a day for about three years. Interferon alpha may also be administered, preferably 15 million units subcutaneously three times a week for about three years. In addition, aspirin is preferably administered daily, preferably about 500 mg with sucralfate three times per day for about five years.

In another aspect, the invention provides a method of identifying a chronic viral infection in a patient and determining a relative prognosis. Blood is isolated from a patient, such as a patient with an acute hepatitis C infection. PBMCs are isolated from the patient's blood. The patient's blood is preferably maintained under conditions to keep it as fresh as possible in order to keep the mitochondria healthy. The mitochondria are isolated by fractionation of the PBMCs. RT-PCR is performed for viral RNA in the mitochondrial fraction. A positive result means that the patient has chronic viral infection, and thus a worse prognosis than a negative result would suggest.

### DETAILED DESCRIPTION OF THE PREFERRED EMBODIMENT

Methods for treating a patient before, during and after transplantation of a donor organ are presented. The methods reduce the chance that the donor organ will be rejected. The methods also treat the underlying disease or disorder, further enhancing the chance of organ acceptance. Although described below in relation to liver transplantation resulting from viral infection or cancer, one of skill in the art will recognize that the methods disclosed herein are applicable to the transplantation of other organs and the underlying disease state.

Preferred methods combine treatments that induce immunotolerance of the transplanted organ while suppressing the immunotolerance of the underlying disease agent, such as a virus or tumor, because the immunotolerance is a main mechanism of the disease. The treatments may be administered simultaneously, or may be administered in a sequential fashion, as discussed below.

A patient suffering from a disease or disorder that necessitates an organ transplant, such as chronic hepatitis or liver cancer, is treated with immunosuppressive agents and agents to treat the underlying disease or disorder. The treatment induces immune tolerance to the donor organ while tolerance of the body to the virus is suppressed.

Donor Organ/Tissue Tolerance

Following transplantation, one or more immunosuppressive agents are administered to the patient to increase immune tolerance to the donor organ/tissue. The type of immunosuppressive agent is not limited in any way, and one of ordinary skill in the art would be aware of immunosuppressive agents that can be used. The immunosuppressive agents are administered in a dosing regimen and at a concentration that is effective for inducing immunosuppression, as can be determined by the skilled practitioner. Preferably they are administered according to established protocols.

In preferred embodiments one or more T cell specific immunosuppressive drugs are used. In a particularly preferred embodiment the T cell specific immunosuppressive drug is Rapamycin (RAPA). Preferably, the T cell specific immunosuppressive drugs do not cause a significant epithelial reaction such as nephrotoxity. Epithelial reaction has been considered one of the most troublesome side effects may result in nephrotoxicity or diabetes. However, this reaction for the epithelial cells in the thymic capsular epithelial cells is a key mechanism of control of immunotolerance. The usual drugs for nephrotoxicity, such as FK 506 or CsA activate the thymic capsular epithelial cell which causes more infiltration and more homing of the carrier cells. This not only enhances graft acceptance, but also enhances the tolerance for viruses or tumors as well. Therefore, in one embodiment, these drugs are not used in an early stage in order to break the tolerance to chronic infection and/or tumor. Rather, RAPA or other drugs with minimal to no epithelial reaction are used for graft acceptance without disturbing control of the tolerance of diseases. Steroids may also act very broadly and in some embodiments are avoided initially during treatment. In one embodiment, after the tolerance for the diseases has been controlled, FK 506 is used for graft acceptance.

In other embodiments, one or more T cell specific immunosuppressive drugs which involve an epithelial reaction are used. In one embodiment, the T cell specific immunosuppressive drug is used after the tolerance to a disease has been controlled. In a further embodiment, the T cell specific immunosuppressive drug is used in a patient without substantial chronic infection and/or cancer. T cell specific immunosuppressive drugs which involve an epithelial reaction include, but are not limited to, Tacrolimus (FK 506), Cyclosporine A, and Imuran (azathioprine). The T cell specific drugs which have an effect on cells of epithelial origin may be used in addition to or as a substitute for the T cell specific drug.

In further embodiments, mixtures of immunosuppressive agents may also be used at any time. In one embodiment, the mixtures are used after the tolerance for the disease has been controlled. In a further embodiment, the mixtures are used during the treatment. For example, and without limitation, the mixtures may include one or more T cell specific immunosuppressive drugs, a T cell specific immunosuppressive drug and a different type of immunosuppressive drug, and a T cell specific drug and a different type of immunosuppressive agent altogether. In a further embodiment, another immunosuppressive drug is administered in combination with the T cell specific drug.

In some embodiments immunosuppressive agents other than drugs are utilized, preferably T cell specific immunosuppressive agents which do not cause a significant epithelial reaction such as nephrotoxity. The agents may be, for example and without limitation, antibodies, preferably monoclonal antibodies. Immunosuppressive antibodies, including T cell specific immunosuppressive antibodies are well known in the art. The antibodies include but are not limited to: anti-CD4 antibodies and anti-CD8 antibodies. In one embodiment, the antibody OKT3 is administered to the patient according to established protocols.

The immunosuppressive drugs and agents are used at a concentration which is effective to control rejection of the donor liver for a time sufficient to facilitate acceptance of the donor liver. The use of the immunosuppressive agents is preferably continued for a time of from about 6 months to 10 years after transplantation, more preferably for a time of from about 1 year to 3 years after transplantation. The amount of time includes but is not limited to 2 months, 5 months, 6 months, 8 months, 12 months, 15 months, 2 years, 3 years, 4 years, 5 years, 7 years, 8 years, and 10 years. Preferably, the amount of time is from about 6 months to about 5 years. The amount of time may vary for each of the immunosuppressive drugs or agents used.

In one embodiment, rapamycin is administered to a patient for a period of five years following transplantation.

In a further embodiment, after about six months to five years of treatment with a T cell suppressive drug which does not effect an epithelial reaction, such as RAPA, another immunosuppressive drug is administered. For example, in one embodiment after five years of treatment with Rapamycin, the patient is treated with tacrolimus for an additional five years.

In a further embodiment, rejection of the donor liver is prevented by inducing graft tolerance. The induction of graft tolerance can be in addition to or as a substitute for the immunosuppressive agents. In one embodiment, the patient is inoculated with about 10⁸ to 10¹⁰ per kg bodyweight donor cells by injection at the time of transplantation or very soon before or after transplanting the new liver. In one embodiment, the donor cells are injected at the time of transplantation or very soon after transplantation. In a further embodiment, the donor cells are injected after transplantation at a time of less than about 12 hours, including but not limited to, less than about 11 hours, 10 hours, 9 hours, 8 hours, 7 hours, 6 hours, 5 hours, 4 hours, 3 hours, 2 hours, and 1 hour. In a further embodiment, the donor cells are injected after transplantation at a time of less than about 1 hour, including but not limited to; 55 minutes, 50 minutes, 45 minutes, 40 minutes, 35 minutes, 30 minutes, 25 minutes, 20 minutes, 15 minutes, 10 minutes, and 5 minutes.

The donor cells may be any donor cell which encodes the immunologic information for graft tolerance, including but not limited to donor peripheral blood mononuclear cells (PBMCs) or donor lymphocytes. Donor lymphocytes are preferably obtained from the spleen, lymph node or blood. The donor cells are maintained in culture as is well known in the art. In other embodiments, the injections of donor cells are repeated until the new graft is accepted. In one embodiment, the injections are repeated every 2-3 weeks for about 6 months, including but not limited to, 1 month, 2 months, 3 months, 4 months, 5 months, and 7 months. In a further embodiment, the injections are repeated every 1 to 4 weeks.

In a particular embodiment, the patient is inoculated intravenously with about 3×10⁸ donor lymphocytes/kg following transplantation. In one embodiment, the patient is inoculated at the time of transplantation and then, about 1 day after transplantation to about 2 months after transplantation, including but not limited to: 2 days, 3 days, 4 days, 5 days, 6 days, 7 days, 10 days, 2 weeks, 3 weeks, 4 weeks, 5 weeks, 6 weeks, 7 weeks, and 8 weeks. In a further embodiment, the cells are injected from about 2 days to about 2 weeks after transplantation.

The immunosuppressive agents and the donor cells can be administered at the same time, at different times, in succession, in combination or in a stepwise fashion. The donor cells can be maintained in the usual culture system and the injection can be repeated until the tolerance of the disease is controlled.

In one embodiment, after the disease is controlled, FK 506 or CsA can be used for inducing graft tolerance.

The methods allow for a quicker and more complete acceptance of the transplanted organ. As a result, it is possible to wean the patient from the immunosuppressive agents at an earlier time than with most traditional methods. Thus, in one embodiment, early weaning from the immunosuppressive drug is effected. In a particular embodiment, the patient is weaned from the immunosuppressive agent at a time period of from about six months to ten years after transplantation, preferably from about one year to about five years after transplantation.

Breaking Immune Tolerance of Chronic Viral Infection

If transplantation has resulted from an underlying viral infection, developing immunotolerance to the transplanted organ is accompanied by methods to break and suppress immunotolerance to the virus. The persistence of many viral infections is mainly due to the tolerance of the immune system to the virus, thus producing a chronic infection. In order to break the immune tolerance to a chronic viral infection, one or more of the following methods is used.

In one embodiment, a patient is treated with interferons, preferably an interferon alpha, preferably for the treatment of hepatitis C infection, beginning just after removal of the old organ. The skilled practitioner will be able to determine the maximum tolerable dosing schedule. The maximum tolerable dose of interferon treatment is preferably continued as long as possible to erase the source which allows the maintenance of immune tolerance of the virus. In one embodiment, interferon treatment is combined with antiviral treatment, using known agents and protocols.

Interferons prevent cells from being infected by many viruses. If a cell is treated with interferon, the interferon changes the properties of that cell and can prevent the virus from replicating in that cell and thus prevent the infection of surrounding cells. It does this by a wide variety of mechanisms that vary from virus to virus and from cell to cell. Interferon-alphas are used to treat viral diseases. At this time there are 12 identified interferon alphas that are approved by the FDA for treatment of chronic hepatitis B and chronic hepatitis C. However, interferon in high doses can cause side effects, such as headache, chills, fever, back pain, joint pain, and anorexia. Thus, in one embodiment, the side effects of interferon are controlled using narcotics with or without respiratory support.

In a preferred embodiment, interferon alpha is administered to the patient subcutaneously three times per week for three years. In one embodiment, the dose is the maximum tolerable dose of the Interferon for that patient and this dose can be readily established by the skilled practitioner for the specific patient. In one embodiment, up to about 15 million units of interferon alpha is administered each time. In a further embodiment, from about 2 million to about 20 million units is administered each time, including but not limited to: 3 million, 4 million, 5 million, 6 million, 7 million, 8 million, 9 million, 10 million, 11 million, 12 million, 13 million, 14 million, 15 million, 16 million, 17 million, 18 million, and 19 million.

In a further embodiment, at least one prostaglandin is administered in order to inactivate the barrier of tolerogenic organs and/or tolerogenic tissues. The tolerogenic organs and tissues are those which are involved in the process of immune tolerance. Preferably, the organ or tissue is an organ or tissue of the immune system, including but not limited to the thymus and the cryptogenic patch.

Prostaglandins are preferably initially administered for a period of time of about three weeks or more following transplantation. Treatment with prostoglandins may be continued indefinitely, preferably for about 5 years. In one embodiment, a patient is treated with a first concentration of prostaglandins for a period of about three weeks by continuous infusion and subsequently treated with a second dose by mouth for an extended period, preferably about 3 years.

Any prostaglandin may be used including but not limited to, PGE1, PGE2, PGI1, and PGI2. A standard dosing regimen is typically employed, including but not limited to the maximum tolerable dose which is accepted by the patient and this dose can be readily established by the skilled practitioner for the specific patient.

In a particular embodiment, after reperfusion of a new liver, a patient receives 32 ug/kg/day of PGE1 by continuous infusion for three weeks, followed by 200 mg by mouth three times per day for three years.

In a further embodiment, to reduce inflammation, at least one nonsteroidal anti-inflammatory drug (NSAID) is also administered to the patient. The skilled practitioner will be able to determine the appropriate dosing regimen. Preferably NSAID treatment is continued for as long as possible to inactivate the barrier of tolerogenic organs/tissues.

Aspirin, preferably with sucralfate, may also be administered to the patient after transplantation. In a preferred embodiment, 500 mg of aspirin with 1.0 g sucralfate is administered to the patient by mouth three times per day for five years following transplantation.

The administration of interferon, antibodies, prostaglandins and NSAIDS may be carried out simultaneously, at different times, in succession, in combination or in a stepwise fashion. Dosages known to one of skill in the art in relation to the treatment chronic viral infection are preferably used.

In other embodiments, passive immunization using antibodies specific for the virus is used to eliminate the source of immune tolerance. Preferably, the antibodies are administered for at least one week, and administration can be continued for an indefinite time (until death). The use of passive immunization prevents immunologically informed carriers from homing into tolerogenic organs and/or tissues.

In one embodiment, the immunoglobulin HBIG is administered to a patient suffering from HBV infection according to known protocols for a period of five years following transplantation to keep the blood HBV antibody level more than 100 IU. In a further embodiment, HBs antibody levels are used to keep the antibody level more than about 150 IU.

In still other embodiments, the patient is treated with a vaccine for the virus, if one is available. For example, in one embodiment an HBV patient is administered the HBV vaccine five years following organ transplantation. Preferably the vaccine is administered after treatment to reduce immunotolerance, as described above.

In a further embodiment, antiviral agents which are effective in treating the virus are used. Examples of particular anti-viral treatments and the associated diseases include, but are not limited to: Acyclovir and related pharmaceuticals which can be used to treat Herpes Simplex virus, anti-retroviral agents which can be used for HIV and other retroviruses, and amantadine, rimantadine, and oseltamivir which can be used to treat influenza. Any newly identified treatments may be used in combination with the methods disclosed herein. Ribavirin can be used according to a dose-escalation schedule to treat hepatitis B. In one embodiment, ribavirin is used at 600 mg/day for the treatment of hepatitis C. In a further embodiment, lamivudine is used at 100 mg daily for the treatment of hepatitis B.

In a further embodiment, the status of immunotolerance to a certain virus is monitored by identifying the presence of one or more of the following: mitochondrial viral nucleic acid in peripheral mononuclear cells (PBMCs), mitochondrial antigens in blood, and mitochondrial antigens in PBMCs. This information can then be used to adjust the schedule of treatment. In one embodiment, if a patient who previously had viral nucleic acid in PBMCs was treated to a point in which no more viral nucleic acid was detectable after transplantation, then Tracrolimus or Cyclosporine A could be used instead of RAPA. Further, if the patient had mitochondrial antigens in his/her blood and/or PBMCs before transplantation, but the antigens were no longer measurable after transplantation, Tacrolimus or Cyclosporine A could be used instead of RAPA.

Breaking Immune Tolerance of a Tumor

It is likely that one of the main reasons for the persistence of cancers is that the immune system has become tolerized to the cancer and/or no longer recognizes the cancer antigens. Thus, if transplantation is necessitated by cancer in a patient, the method herein involves the process of developing immunotolerance to the transplanted organ accompanied by methods to reduce immunotolerance to the cancer cells. Thus, in another aspect of the invention, recurrence of a tumor after transplantation is prevented by breaking immune tolerance to the cancer while inducing immune tolerance for the newly transplanted organ.

Induction of immune tolerance for the transplanted organ is carried out as described above.

In one embodiment, to remove immune tolerance to the cancer, a patient is treated with interferons, preferably an interferon alpha, beginning just after reperfusing the grafted organ. The skilled practitioner will be able to determine the appropriate dosing schedule. Interferon treatment is preferably continued as long as possible to erase the source which allows the maintenance of immune tolerance of the cancer. In one embodiment, interferon treatment is combined with an anticancer treatment, using known agents and protocols.

Interferon works in a different way toward cancer cells than it does toward viruses and there are numerous pathways that interferon activates to help treat cancers. It has an antiproliferative effect on tumor cells, it stimulates the tumor cells to change their surfaces so that they are recognized by the immune system as abnormal cells, and it blocks the growth of new blood vessels and helps cut off the supply of nutrients. At this time there are 12 identified interferon alphas.

Interferon in high doses can cause side effects, such as headache, chills, fever, back pain, joint pain, and anorexia. Thus, in one embodiment, the side effects of interferon are controlled using narcotics with or without respiratory support.

Prostaglandins are preferably administered for a period of time of about three weeks or more following transplantation. Any prostaglandin may be used including but not limited to, PGE1, PGE2, PGI1, and PGI2. A standard dosing regimen is typically employed, including but not limited to the maximum tolerable dose which is accepted by the patient as can be readily established by the skilled practitioner.

In a particular embodiment, after reperfusion of a new liver, a patient receives 32 ug/kg/day of PGE1 by continuous infusion for three weeks, followed by 200 mg by mouth three times per day for three years.

In a further embodiment, to reduce inflammation, at least one nonsteroidal antinflammatory drug (NSAID) is also administered to the patient. The skilled practitioner will be able to determine the appropriate dosing regimen. Preferably NSAID treatment is continued for long as possible to inactivate the barrier of tolerogenic organs/tissues.

Aspirin, preferably with sucralfate, may also be administered to the patient after transplantation. In a preferred embodiment, 500 mg of aspirin with 1.0 g sucralfate is administered to the patient by mouth three times per day for five years following transplantation.

In other embodiments, passive immunization using antibodies specific for the cancer or at least one antigen associated with the cancer is used to eliminate the source of immune tolerance. Preferably, the antibodies are administered for at least one week, and administration can be continued for an indefinite time (until death). The use of passive immunization prevents immunological informed carriers from homing into tolerogenic organs and/or tissues.

The administration of interferon, antibodies, prostaglandins and NSAIDS may be carried out simultaneously, at different times, in succession, in combination or in a stepwise fashion. Dosages known to one of skill in the art in relation to the treatment of chronic viral infections are preferably used.

### EXAMPLES

Further details of the invention are illustrated in the following non-limiting examples.

### Example 1

Thymic Homing Control Using Tacrolimus or Rapamycine

Inbred male Lewis rats (LW, RT1l) were injected intramuscularly with rapamycine (RAPA; 0.1 mg/kg/d), tacrolimus (TAC; 0.2 mg/kg/d), cyclosporine A (CsA; 10 mg/kg), PGE 1 (32 ug/kg or 10 ug/kg) or a control.

One hour after the above treatments, 1.2-1.6×10⁷ peripheral blood mononuclear cells (PBMCs) were obtained from Brown Norway rats (BN, RT1n). The cells were stained with IUBD and then injected intraperitoneally. 24 hours after cellular inoculation, the animals were sacrificed. A thymectomy was performed, the thymus was sliced and homed cell counting was performed. Homed cells may be counted by any method known to one of skill in the art. In this case, as the cells were stained before inoculation, the homed cells in the thymus were identified by the staining. The results of the cell counting are presented in Tables 1 and 2 for each treatment group:

TABLE 1

| | FK506 | CsA | RAPA | control |
|---|---|---|---|---|
| Donor Cells injected. | 1.5 × 10⁷ | 1.6 × 10⁷ | 1.2 × 10⁷ | 1.6 × 10⁷ |
| Thymic homing (cell | 2360 | 3365 | 920 | 6580 |
| number) | | | | |

TABLE 2

| PGE1 | 32 ug/kg | 10 ug/kg | control |
|---|---|---|---|
| Donor Cells injected | 1.6 × 10⁷ | 1.6 × 10⁷ | 1.6 × 10⁷ |
| Thymic homing (cell number) | 4680 | 8890 | 6580 |

FK, CsA, and RAPA all inactivated PBMCs that homed to the thymus, as more homing was seen in the control group. Of the treated groups, the RAPA group showed much less homing than FK or CsA. RAPA does not activate the epithelial cells of the thymus, whereas, FK and CsA do activate the epithelial cells of the thymus.

Further, a high dose (32 ug/kg) of PGE1 inactivated the epithelial cells as well as the PBMCs. The high dose showed less thymic homing than low doses and also showed less homing than in the control.

### Example 2

Tumor Transplantation

Inbred male C57BL/6Cr mice were used. Peripheral blood mononuclear cells (PBMCs) were isolated in the usual manner from both inbred rats which had previously accepted Lewis lung carcinoma cells (tumor accepted) and from normal mice (control). Both types of PBMCs were inoculated at a level of 10⁸ cells into other mice. 10 days after inoculations, 2×10⁶ Lewis lung carcinoma cells were inoculated subcutaneously. The growth of tumors was assessed and the results are shown in Table 3.

TABLE 3

| PBMC from | tumor acceptance | % |
|---|---|---|
| PBMC from tumor accepted | 13/14 | 92.9 |
| control PBMC | 11/15 | 73.3 |
| no PBMC | 6/10 | 60.0 |

Further, if PBMCs from tumor accepted animals were inoculated less than 5 days before tumor graft, the cells did not affect tumor acceptance. This suggests that PBMCs do not affect tumor tolerance in a direct way, but in a more indirect way which requires more time.

The following examples provide exemplary clinical protocols. Each specific treatment with a specific drug is performed according to protocols accepted in the field unless otherwise specified. Dosages and frequencies are also as known to one of skill in the art unless otherwise indicated.

### Example 3

### HBV After Transplantation

A patient suffering from HBV undergoes a liver transplant. After reperfusion of the new liver, the patient is treated as follows. Rapamycin is administered for 3 to 5 years with no concomitant steroid treatment. Donor lymphocytes obtained from the spleen, lymph node or blood are injected at a concentration of 3×10⁸ cells/kg intravenously. Hepatitis B immune globulin (HBIG) is administered according to established procedures to keep the blood HBsAg antibody level at more than 150 IU for 5 years.

The status of immunotolerance to HBV is monitored by identifying the presence of mitochondrial HBV genes in PBMC and/or mitochondrial antigens in blood. This information is used to adjust the schedule of treatment.

After about 3 years the patient is weaned of the immunosuppressive agent (rapamycin) with close monitoring of liver function. If necessary, treatment with a second immunosuppressive agent, such as tacrolimus, is begun and continued for 5 years, after which time the patient is weaned. Rejection is controlled with OKT3, which is administered according to standard protocols. The HBV vaccine is administered about 5 years after transplantation.

### Example 4

### HCV After Transplantation

A patient suffering from HCV undergoes a liver transplant. After reperfusion of the new liver, the patient is treated as follows. Rapamycin is administered for about 5 years with no concomitant steroid treatment. Donor lymphocytes obtained from the spleen, lymph node or blood are injected at a concentration of 3×10⁸ cells/kg intravenously. PGE 1 (32 ug/kg/day) is administered by continuous infusion for about 3 weeks, followed by 200 mg by mouth three times per day for about 3 years. Interferon alpha (about 15 million units) is administered subcutaneously three times per week for about 3 years. Aspirin (500 mg) with sucralfate (1.0 g) is administered by mouth 3 times per day for about 5 years.

The status of immunotolerance to HCV is monitored by identifying the presence of mitochondrial HCV genes in PBMCs and/or mitochondrial antigens in blood. This information is used to adjust the schedule of treatment.

After about 5 years the patient is weaned of the immunosuppressive agent (rapamycin) with close monitoring of liver function. If necessary, treatment with a second immunosuppressive agent, such as tacrolimus, is begun and continued for 5 years, after which time the patient is weaned. Rejection is controlled with OKT3, which is administered according to standard protocols.

### Example 5

### HIV After Transplantation

A patient suffering from HIV undergoes a liver transplant. After reperfusion of the new liver, the patient is treated as follows. Rapamycin is administered for about 5 years with no concomitant steroid treatment. Donor lymphocytes obtained from the spleen, lymph node or blood are injected at a concentration of 3×10⁸ cells/kg intravenously. PGE 1 (32 ug/kg/day) is administered by continuous infusion for about 3 weeks, followed by 200 mg by mouth three times per day for about 3 years. Any anti-HIV treatment is used which is known to one of skill in the art. Aspirin (500 mg) with sucralfate (1.0 g) is administered by mouth 3 times per day for about 5 years.

The status of immunotolerance to HIV is monitored by identifying the presence of mitochondrial HIV genes in PBMCs and/or mitochondrial antigens in blood. This information is used to adjust the schedule of treatment.

After about 5 years the patient is weaned of the immunosuppressive agent (rapamycin) with close monitoring of liver function. If necessary, treatment with a second immunosuppressive agent, such as tacrolimus, is begun and continued for 5 years, after which time the patient is weaned. Rejection is controlled with OKT3, which is administered according to standard protocols.

### Example 6

### Hepatoma Treatment After Transplantation

A patient suffering from a hepatoma undergoes a liver transplant. After reperfusion of the new liver, the patient is treated as follows. Rapamycin or another immunosuppressive agent is administered for about 5 years with no concomitant steroid treatment. Donor lymphocytes obtained from the spleen, lymph node or blood are injected at a concentration of 3×10⁸ cells/kg intravenously. PGE 1 (32 ug/kg/day) is administered by continuous infusion for about 3 weeks, followed by 200 mg by mouth three times per day for about 3 years. Aspirin (500 mg) with sucralfate (1.0 g) is administered by mouth 3 times per day for about 5 years.

The status of immunotolerance to hepatoma is monitored by examining any mitochondrial abnormality of PBMCs as compared to a control. The control can be one or both of the original tumor tissue and PBMCs before treatment of the tumor tissue. The status may be used to analyze treatment and in order to adjust the schedule of treatment.

After about 5 years the patient is weaned of the immunosuppressive agent (rapamycin) with close monitoring of liver function. If necessary, treatment with a second immunosuppressive agent, such as tacrolimus, is begun and continued for 5 years, after which time the patient is weaned. Rejection is controlled with OKT3, which is administered according to standard protocols.

### Example 7

### Prognostic Diagnosis of HCV Infection—Early Detection Using a Viral Gene in PBMCs

Blood is isolated from an hepatitis C patient with an HCV infection. PBMCs are isolated from the patient's blood. The patient's blood is kept as fresh as possible to keep the mitochondria healthy. The mitochondria are isolated by fractionation of the PBMCs. RT-PCR is performed for HCV RNA in the mitochondrial fraction. A positive result means that the patient has chronic hepatitis C with a worse prognosis than a negative result would suggest. A negative result means that the hepatitis C patient has an acute infection.

### Example 8

### Method of Treatment Including Monitoring of the Continued Presence of a Viral Infection and/or Cancer

The viral gene in the mitochondria of PBMC's is monitored before and after transplantation and/or other treatment. When the viral gene is substantially absent from the mitochondria, the central tolerance of the viral infection is likely broken. From that point aggressive immunosuppression to allow graft acceptance is initiated using a T cell specific immunosuppressant with a possible epithelial reaction, such as tacrolimus or cyclosporine.

For cancer patients, mitochondrial genomic abnormality is monitored before and after transplantation; If a preexisting mitochondrial genomic abnormality before treatment has disappeared or is substantially absent after treatment and/or after transplantation, one can assume that the central tolerance to the tumor has been removed and the treatment was successful. At this point, the patient is diagnosed as “in remission” or cured of cancer. At this point also, aggressive immunosuppression to allow graft acceptance is initiated using T cell specific immunosuppressants (with epithelial reactions), such as tacrolimus or cyclosporine during or prior to transplantation.

Mitochondrially translated viral proteins, virus-related proteins, tumor proteins, tumor related proteins or other disease-related proteins are analyzed by comparing protein profiles from cells containing viral genes, or other abnormal genes in the mitochondria with normal cells which are blocked in mitochondrial translation.

Methods of blocking mitochondrial translation include any of those known in the arts. For example, nonspecific methods of blocking mitochondrial translation are known in the arts, as are specific methods of blocking, for example, by antisense technologies.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

What is claimed is:

1. A method of controlling chronic viral infection and enhancing acceptance of a donor organ in a transplant patient with a chronic viral infection, comprising:
administering an antiviral agent to the patient; and
administering an immunosuppressive agent to the patient.

2. The method of claim 1 wherein said immunosuppressive agent is an immunosuppressive agent which does not cause an epithelial reaction.

3. The method of claim 1 wherein said immunosuppressive agent is a T cell specific immunosuppressive agent which does not cause an epithelial reaction.

4. The method of claim 3, wherein the T cell specific immunosuppressive agent is rapamycin (RAPA).

5. The method of claim 3, wherein the T cell specific immunosuppressive agent is an anti-T cell antibody.

6. The method of claim 5 wherein the anti-T cell antibody is selected from the group consisting of an anti-CD4 antibody and an anti-CD8 antibody.

7. The method of claim 2, wherein the immunosuppressive agent which does not cause an epithelial reaction is Imuran.

8. The method of claim 2 and 3, wherein a different immunosuppressive agent is administered to the patient after about 6 months to five years of treatment with the T cell specific or nonspecific immunosuppressive agent which does not include an epithelial reaction.

9. The method of claim 1, wherein the immunosuppressive agent is administered after transplantation.

10. The method of claim 1, wherein the immunosuppressive agent is administered both before and after transplantation.

11. The method of claim 1, wherein administration of the immunosuppressive agent is continued for a period of from about 6 months to ten years after transplantation.

12. The method of claim 1 further comprising administering donor cells to the patient.

13. The method of claim 12, wherein the donor cells are peripheral blood mononuclear cells.

14. The method of claim 12, wherein from about 108 to about 109 donor cells per kg body weight of the patient are administered to the patient.

15. The method of claim 12, wherein the donor cells are administered during transplantation.

16. The method of claim 11, wherein the donor cells are administered from about 1 day to about 60 months after transplantation.

17. The method of claim 1 further comprising administering an effective amount of a second immunosuppressive agent to the patient.

18. The method of claim 1 wherein said antiviral agents are administered during and after transplant.

19. The method of claim 1 wherein said antiviral agents and said immunosuppressive agents are administered simultaneously.

20. The method of claim 1 wherein said antiviral agents and said immunosuppressive agents are administered in succession.

21. The method of claim 1 wherein said antiviral agent is interferon alpha.

22. The method of claim 1, wherein said antiviral agent is an antibody specific for the virus.

23. The method of claim 1, further comprising administering a prostaglandin to the patient following transplantation.

24. The method of claim 23, wherein the prostaglandin is selected from the group consisting of PGE1, PGE2, PGI1 and PGI2.

25. The method of claim 23, wherein administration of the prostaglandin is continued for a period of at least about three weeks following transplantation.

26. The method of claim 1, further comprising administering a nonsteroidal anti-inflammatory drug to the patient following transplantation.

27. The method of claim 1, wherein said donor organ is a liver.

28. The method of claim 1, wherein said chronic viral infection is hepatitis virus infection.

29. The method of claim 28, wherein the hepatitis virus is selected from the group consisting of hepatitis A, hepatitis B, hepatitis C and hepatitis D virus.

30. The method of claim 1, further comprising monitoring the cells for the presence of the virus.

31. The method of claim 30, further comprising altering the treatment based on the results of monitoring.

32. A method of preventing the recurrence of cancer and enhancing acceptance of a donor organ transplant in a patient with cancer, comprising:
administering an immunosuppressive agent to said patient; and
administering an anti-cancer agent to said patient.

33. The method of claim 32 wherein said immunosuppressive agent is an immunosuppressive agent which does not include an epithelial reaction.

34. The method of claim 32 wherein said immunosuppressive agent is a T cell specific immunosuppressive agent which does not include an epithelial reaction.

35. The method of claim 34, wherein the T cell specific immunosuppressive agent is rapamycin (RAPA).

36. The method of claim 34, wherein the T cell specific immunosuppressive agent is an anti-T cell antibody.

37. The method of claim 36 wherein the anti-T cell antibody is selected from the group consisting of an anti-CD4 antibody and an anti-CD8 antibody.

38. The method of claim 33 wherein the immunosuppressive agent is Imuran.

39. The method of claim 33 and 34, wherein a different immunosuppressive agent is administered to the patient after about 6 months to five years of treatment with the T cell specific or nonspecific immunosuppressive agent which does not include an epithelial reaction.

40. The method of claim 32, wherein the immunosuppressive agent is administered during and after transplantation.

41. The method of claim 32, wherein the immunosuppressive agent is administered both before, during and after transplantation.

42. The method of claim 32, wherein administration of the immunosuppressive agent is continued for a period of from about 6 months to ten years after transplantation.

43. The method of claim 32 further comprising administering donor cells to the patient.

44. The method of claim 43, wherein the donor cells are peripheral blood mononuclear cells.

45. The method of claim 43, wherein from about 108 to about 109 donor cells per kg body weight of the patient are administered to the patient.

46. The method of claim 32 further comprising administering an effective amount of a second immunosuppressive agent to the patient.

47. The method of claim 32 wherein said anticancer agents are administered during and after transplant.

48. The method of claim 32 wherein said anticancer agents and said immunosuppressive agents are administered simultaneously.

49. The method of claim 32 wherein said anticancer agents and said immunosuppressive agents are administered in succession.

50. The method of claim 32 wherein said anticancer agent is interferon alpha.

51. The method of claim 32, wherein said cancer agent is an antibody specific for the cancer.

52. The method of claim 32, further comprising administering a prostaglandin to the patient following transplantation.

53. The method of claim 52, wherein the prostaglandin is selected from the group consisting of PGE1, PGE2, PGI1 and PGI2.

54. The method of claim 52, wherein administration of the prostaglandin is continued for a period of at least about three weeks following transplantation.

55. The method of claim 32, further comprising administering a nonsteroidal anti-inflammatory drug to the patient following transplantation.

56. The method of claim 32, wherein said donor organ is a liver.

57. The method of claim 32, wherein said cancer is liver cancer.

58. A method of treating a patient suffering from hepatitis B (HBV) following a liver transplant comprising:
administering rapamycin to the patient;
injecting donor lymphocytes intravenously; and
administering hepatitis B immune globulin (HBIG);

59. The method of claim 58, further comprising administering OKT3 for the control of rejection.

60. The method of claim 58, wherein about 3×10⁸/kg donor lymphocytes are administered.

61. A method of treating a patient suffering from a disease or disorder following a liver transplant comprising:
administering rapamycin to the patient for five years;
injecting donor lymphocytes intravenously;
administering PGE1 for about three weeks;
administering interferon alpha three times per week for about three years; and
administering OKT3 for rejection control.

62. The method of claim 61, wherein administering PGE1 comprises administering about 32 ug/kg/day by continuous infusion.

63. The method of claim 61, wherein about 3×10⁸/kg donor lymphocytes are administered.

64. The method of claim 61, wherein about 15 million units of interferon alpha is administered three times per week for about three years.

65. The method of claim 61, additionally comprising administering aspirin about three times a day for about five years.

66. The method of claim 61, wherein the disease is selected from the group consisting of hepatitis C virus infection and a hepatoma.

67. A method for identifying whether a viral disease in a patient is chronic or acute, comprising,
identifying the presence of said virus in the mitochondria of said patient's cells,
wherein the presence in mitochondria is associated with chronic infection.

68. A method of enhancing acceptance of a donor organ in a patient with a viral infection, comprising:
testing mitochondria for the presence of viral nucleic acid prior to transplantation;
testing mitochondria for the presence of viral nucleic acid after transplantation; and
if viral nucleic acid is not present, administering a T cell specific immunosuppressive agent with an epithelial reaction.

69. The method of claim 68 wherein the T cell specific immunosuppressive agent is tacrolimus.

70. The method of claim 68 wherein the T cell specific immunosuppressive agent is cyclosporine.

71. The method of claim 68 wherein testing comprises comparing proteins from cells in which mitochondrial translation has been blocked to cells in which mitochondrial translation has not been blocked.

72. A method of enhancing acceptance of a donor organ in a patient with cancer comprising:
testing mitochondria for the presence of a genomic abnormality prior to transplantation;
testing mitochondria for the presence of a genomic abnormality after transplantation; and
if a genomic abnormality is not present, administering a T cell specific immunosuppressive agent with an epithelial reaction.

73. The method of claim 72 wherein the T cell specific immunosuppressive agent is tacrolimus.

74. The method of claim 72 wherein the T cell specific immunosuppressive agent is cyclosporine.

71. The method of claim 72 wherein testing comprises comparing proteins from cells in which mitochondrial translation has been blocked to cells in which mitochondrial translation has not been blocked.
